(19) United States
(12) Patent Application Publication
(10) Pub. No.: US 2004/0029172 A1
(43) Pub. Date: Feb. 12, 2004

(54) Generation of combinatorial libraries and assessment thereof by deconvolution

(76) Inventors: Jean-Marie Lehn, Strasbourg (FR); Olof Ramstrom, Stockholm (SE); Taridoporn Bunyapaiboonsri, Strasbourg (FR)

Correspondence Address: BURNS DOANE SWECKER & MATHIS L L P, POST OFFICE BOX 1404, ALEXANDRIA, VA, 22313-1404, US

(21) Appl. No.: 10/363,638

(22) PCT Filed: Sep. 4, 2001
(86) PCT No.: PCT/EP01/10170

(30) Foreign Application Priority Data
Sep. 4, 2000 (EP) 00119120.4

(51) Int. Cl. G01N 33/53; G01N 33/543

(52) U.S. Cl. 435/7.1; 436/518

(57) ABSTRACT

The present invention deals with a method for the generation of a dynamic combinatorial library of ligands for a given target, capable of binding at least two functionalities, which method comprises the following steps: (i) selecting a set of n molecules carrying functionalities which may bind to the target, which molecules are capable of reversibly binding to each other under formation of an entity, n being an integer ≧1: (ii) mixing together said set of said n molecules: (iii) subjecting the mixture to conditions allowing a reversible bond formation and cleavage under formation of entities carrying different combinations of functionalities, until equilibrium is reached: (iv) optionally transforming the said entities into ligands: (v) repeating steps (i) to (iv) n-times, each time a different molecule of said set of n molecules being omitted: (vi) assessing the ability of each of the mixtures obtained to bind to a given target.

The present invention relates to a method for the generation of combinatorial libraries (CLs) and the assessment of the relative activity of a given functionality or a combination of at least two functionalities. The said assessment of the relative activity is carried out by means of the co-called dynamic deconvolution.

Biologically active compounds and medical drugs were traditionally generated by conventional chemical or biological methods by variation of so-called lead compounds. Since the generation of these compound variants is time-consuming, only a relatively small pool (a small library) of these compound variants is accessible for biological tests.

These problems can be overcome by combinatorial chemistry (CC), a method where a set consisting of various basis components is used to generate a variety of new compounds by successive and repetitive application, of specific chemical reactions. The covalent nonreversible connections between the components are performed individually in parallel or concertedly in the same compartment(s). So, vast combinatorial libraries (CLs) of extensive collections of constituents are available in a very short time. CC is therefore a very powerful method for exploring the molecular geometrical and interactional spaces through molecular diversity generation and is thus based on large populations of different molecules that are present as discrete entities.

In contrast to this, the so-called dynamic combinatorial chemistry (DCC) is a conceptually different approach that requires a reversible assembly process and its constituents may be either molecular or supramolecular.

DCC thus relies on reversible reactions or interactions between sets of basic components to give access to virtual combinatorial libraries (VCLs) of potential entities, allowing the target-driven generation or amplification of the active constituents of the libraries. It thus represents a self-screening process by which the desired optimal species are preferentially expressed and retrieved from the VCL. DCC extends beyond static combinatorial chemistry (SCC) towards adaptive/evolutive chemical systems. It is especially useful for the search of ligands which bind to targets like enzymes, receptors or antibodies. Suitable detected ligands may be used as medicaments.

In Proc. Natl. Acad. Sci. USA 1997, 94, 2106 - 2110 I. Huc and J.-M. Lehn disclose a method for the generation of a dynamic combinatorial library of imines from one set of aldehydes and one set of amines. The method is directed toward the synthesis of inhibitiors of the enzyme carbonic anhydrase by recognition-involved assembly. The synthesis of the above mentioned imines is carried out either in the presence of said enzyme (so-called adaptive combinatorial libraries) or said enzyme is added after equilibration (so-called pre-equilibrated dynamic or post-dynamic combinatorial libraries, pDCLs).

The non-published PCT-Application carrying the application number PCT/EP01/02310 of the applicant discloses the in situ generation and screening of a dynamic combinatorial carbohydrate library against Concanavalin A. The plant lectin Concanavalin A belongs to the broad class of carbohydrate binding proteins. It is either present during library generation or is added after equilibration.

In both cases, the compounds formed in the generation of the library have to be isolated and separated before they are analyzed by comparison with reference substances or the known analytical methods. This is often time-consuming or may even be impossible. The same often applies to inhibitors generated through static combinatorial libraries.

In order to avoid these drawbacks, there is a strong need for a simple efficient screening of a VCL or an SCL (static combinatorial library) and a rapid identification of active compounds/constituents. This problem is solved by the method of this invention. This method allows the generation and easy identification of substrates, inhibitors, receptors, catalysts and carriers for a variety of processes by means of virtual combinatorial libraries.

The present invention relates to a method for the generation and screening of dynamic combinatorial libraries of ligands. One method comprises the following steps:

(i) selecting a set of n molecules carrying functionalities which may bind to the target, which molecules are capable of reversibly binding to each other under formation of an entity, n being an integer ≧1;

(ii) mixing together said set of said n molecules;

(iii) subjecting the mixture to conditions allowing a reversible bond formation and cleavage under formation of entities carrying different combinations of functionalities, until equilibrium is reached;

(iv) optionally transforming the said entities into ligands;

(v) repeating steps (i) to (iv) n-times, each time a different molecule of said set of n molecules being omitted;

(vi) assessing the ability of each of the mixtures obtained to bind to a given target.

Another method comprises the following steps:

(i) selecting a set of n molecules carrying functionalities which may bind to the target, n being an integer ≧1, and linking paid functionalities by a spacer group, hence creating a set of entities carrying different combinations of said functionalities;

(ii) mixing together said set of entities;

(iii) subjecting the mixture to conditions allowing a reversible bond formation and cleavage of or at the spacer group, hence a scrambling of the functionalities under formation of new entities;

(iv) optionally transforming said entities generated to ligands;

(v) repeating steps (i) to (iv) several times, each time a different functionality of said n functionalities being omitted;

(vi) assessing the ability of each of the mixtures obtained after carrying out the above steps to bind to a given target.

According to the present invention, the terms used herein have the following meanings:

The method according to the present invention can also be used to generate a dynamic combinatorial library of targets. The method then includes the same steps as in the finding of ligands laid but above, but wherein targets are used in place of ligands and vice versa.

“Functionality” means any polar, nonpolar, hydrophilic or lipophilic, or charged unit or subunit or electron donor or electron acceptor group. “Functionality” on the one hand includes, simple functionalities like amino and imino groups and derivatives thereof, hydroxy and mercapto groups and derivatives thereof, oxo and thioxo groups, formyl and thioformyl groups, aryl groups, substituted aryl groups; phenyl groups, substituted phenyl groups, pyridyl groups and derivatives thereof, carboxy groups and carboxylato groups and derivatives therof, alkyloxycarbonyl groups, (di)thiocarboxy groups and derivatives thereof, (di)thiocarboxylato groups, carbamoyl groups and derivatives thereof, sulfo, sulfino and sulfeno groups and derivatives thereof, alkyloxysulfonyl, alkyloxysulfinyl and alkyloxysulfenyl groups, sulfamoyl, sulfinamoyl and sulfenamoyl groups and derivatives thereof, cyano and (iso)(thio)cyanato groups, hydroperoxy groups, nitroso groups, hydroxyamino groups, hydrazino groups, —NR₁R₂, —⁺NHR₁R₂ and —⁺NR₁R₂R₃ groups, wherein R₁, R₂, and R₃ are identical or different and represent alkyl, cycloalkyl, alkylcycloalkyl, aryl, alkylaryl with 1 to 40 C atoms, —⁺OR₁R₂ groups wherein R₁ and R₂ are identical or different and represent alkyl, cycloalkyl, alkylcycloalkyl, aryl, alkylaryl with 1 to 40 C atoms, hydrazide groups and any other suitable groups known to a person skilled in the art.

On the other hand, “functionality” also includes more complex components, and non-limiting examples include heterocycles carrying one or more heteroatoms in the ring selected from the group consisting of N, O and S, amino acids and oligo- and polypeptides, sugars (preferably hexoses and pentoses), sugar derivatives (like peracetylated sugars) and oligomers and polymers therof, and nucleic acids and derivatives thereof.

The functionalities can thus be linked to a molecule, i.e. a molecule carrying the functionality or the functionalities is formed; the functionality can also itself be the molecule (and thus form the molecule carrying the functionality). An example thereof is a sugar molecule

These molecules, i.e. those carrying the functionality or, respectively, those being the functionality itself can furthermore carry one or more functional groups in order to allow reversible bond formation with functional groups present on another molecule, see below.

When the components of the dynamic combinatorial library combine with each other, “entities” are formed. These entities may already present the “ligands” having at least two functionalities to bind to a target. They may also only present precursors of the ligands. That means that another reaction must take place to transform the entities to the ligands like, for example, the reduction of generated imines to amines. The entities may also represent the “targets”.

“Ligand” means a molecule with a molecular weight typically not greater than 1500, preferably not greater than 1000, advantageously not greater than 500, which possesses an affinity for a target, i.e., that is able to interact with the target by forming one or a plurality of weak bonds such as hydrogen bonds, hydrophobic interactions, charge-charge interactions, Van der Waals interactions, donor-acceptor interactions, charge-transfer interactions, metal ion bindings, etc. The ligands generally have at least two functionalities being able to interact with the “target”.

“Target” means a biological or synthetical macromolecule with a molecular weight typically greater than 5000. Biological macromolecules may be proteins including lipoproteins, glycoproteins and analogues of proteins, wherein either the peptide bond —CO—NH— is replaced by an analogous bond, possibly reversible such as an imine, ester, sulfonamide, sulfone, sulfoxide, phosphate, phosphonate, phosphonamide, guanidine, urea, thiourea, or imide bond, or wherein the aminoacids are replaced by synthetical derivatives thereof. The natural proteins may have differing functions, they may act namely as enzymes, as receptors or as antibodies. Receptors may be membrane receptors, hormone receptors, or signal transducers.

If the target is an enzyme, the ligand which is sought to be obtained may act as a substrate, an inhibitor or an activator for said enzyme.

If the target is a receptor, the ligand which is sought to be obtained may act as a natural or artificial ligand, an agonist or an antagonist for said receptor.

If the target is an antibody, the ligand which is sought to be obtained may act as an antigen for said antibody.

If the target is an antigen, the ligand which is sought to be obtained may act as an antibody for said target.

The scope of the present invention is not limited to biological targets. Any natural of synthetical organic and inorganic target may be used. In general, any kind of target for which an activity assay exists, is suitable. The activity may e.g. be determined, by measuring the change of fluorescence, viscosity, conductivity or IR or UV absorption. Therefore suitable targets, besides those cited above may be zeolithes, clathrates, oligonucleotides, oligopeptide, oligosaccharides, sensors, clusters, RNA aptamers, organic and inorganic catalysts, ionophores, any kind of macrocycles like metallomacrocycles, macrocyclic lactams, macrocyclic esters, macrobicyclic cryptands and macrocyclic oligocholates, any kind of synthetic polymers like polyamioacids, polyamides, polyesters, polyalcohols and mixtures thereof, etc. Ligands may have any kind of functional groups mentioned above. Even simple “molecules” like cations or anions may act as ligands. In general, all kinds of molecules, the one of which can act as a ligand and the other one as a target, are suitable to be used in the method according to the present invention.

“Reversible” refers to bond formation and cleavage in a dynamic equilibrium. Reversible bonds not only include reversible covalent reactions, but also reversible connection processes involving non-covalent interactions, such as metal ion coordination, π-stacking, hydrogen bonding, or charge-charge interactions.

Reversible covalent reactions are especially suited, since bond formation and cleavage may occur in particular conditions, and may be inhibited in others. The reversible reaction/connection should take place in or near physiological conditions. In general, the amount of the thermodynamically most stable (supramolecular) species will be the largest amount. Examples for functional groups which may react with other functional groups under reversible bond formation, and wherein the functional groups are present in the molecules carrying the functionalities, include amino groups, aldehyde groups, keto groups, thiol groups, olefinic groups, alcohol groups, carbonyl groups, hydrazine groups, hydroxylamine groups and borate groups. Examples of reversible covalent reactions with the above-mentioned groups involved are those where carbonyl groups react under the formation of imines, acyl-hydrazones, amides, acetals, and esters. In particular, the reaction of amino groups with carbonyl groups to imines, oximes or hydrazones is useful. Reactions such as thiol exchange in disulphides or alcohol exchange in borate esters are further examples, as well as reversible Diels-Alder and other thermal- or photoinduced rearrangements like sigmatropic and electrocyclic rearrangements, and Michael reactions or alkene metathesis using catalysts that may be soluble in water. Photoinduced interconversions represent another possibility leading to photodynamic combinatorial processes.

Dynamic libraries can also be of conformational or configurational character: For example in cis-trans isomerisation, where the difference in configuration can be used in the selection. Conformational dynamic processes are e.g. internal rotation or ring and site inversion. Other suitable library components, reversible processes and biological targets will be known to a person skilled in the art.

The method according to the present invention preferably makes use of pre-equilibrated dynamic combinatorial libraries (pDCLs): The constituents of the library are generated by reversible inter-conversion and equilibration in the absence of a target. After the equilibrium has been reached, the dynamic process is stopped. This may be attained, for example, by changing the reaction conditions. If appropriate, the target can be present in the course of the generation of the library, this being not a preferred embodiment.

After reversibility has been stopped, a target is added. According to known methods this step was carried out in order to “fish out” the suitable ligands and to analyze these after release from the target. In contrast to this, in the methods according to the present invention the target is added in order to assess the ability of the mixture of ligands to bind to this target and is preferably added after equilibrium has been reached.

If the ligand is an inhibitor and the target is an enzyme, assessing the ability of the ligand to bind to the target can be achieved by simply adding the enzyme to the mixture and measuring the enzyme activity. This method works with every protein catalyzing a reaction, that means acting as an enzyme, so that the measurement of enzyme activity is possible. In general, the method of the present invention can be used for any protein for which the activity assay is known. In general, the method of the present invention requires low amounts of target compared to the amounts of the library and lower amounts as in the known methods (adaptive or pre-equilibrated DCLs). This ability can be assessed by any suitable method known to a person skilled in the art. As an example, the measurement of enzyme activity can be done by the Ellmann's test. The inhibition of enzyme activity by a library indicates the presence of one or several active ligands in a given equilibrated mixture.

The relative ability of a given functionality to bind to the target is then assessed by the dynamic deconvolution. According to this principle, the dynamic library is again generated, under conditions identical to those used in the generation of the first library; however, in this second library generation, one of the previously used components will be omitted, i.e. will not be present in the course of the library generation under equilibrium conditions. The reversibility is then again stopped, and the ability of the thus-obtained mixture to interact with the target is measured by known methods. This procedure is then repeated several times, each time omitting one component which was present in the original generation of the library. The ability of each newly-generated library to interact with the target is measured.

Hence, when the original library is generated from n starting molecules with n being an integer >1, the procedure is repeated n times with n-1 starting molecules, thus generating n new libraries and one original library. The abilities of the mixtures obtained (after reversibility has been stopped) to bind to the target are then compared.

If the binding ability of one of the newly generated library is greater, this indicates that the functionality present in the component omitted in the generation of this library does not contribute significantly to the functionality present in the inhibitory effect. A decrease in the binding ability will indicate that the removed component is an important element in the generation of an active constituent in the dynamic mixture. The component, the omitting of which results in the largest decrease in the binding ability, belongs to the best inhibitor.

This procedure amounts to a “dynamic de-convolution”, talking advantage of the dynamic features of the library, since by removal of a given component the remaining components will redistribute and all constituents containing this component will automatically be deleted from the equilibrating library. Such a procedure provides an efficient way to converge on the active constituent(s) of a DCL. Not only in more complex cases with many more library members, but also for getting a ranking of the inhibitors, the procedure may go through successive steps, the experiments with removal of a single component being followed by tests involving the removal of two or more components, thus rendering the procedure convergent.

The principle of the dynamic deconvolution can even be used to establish a ranking list of the functionalities which are present in the components of the starting compounds. In order to further assess the ability of the different functionalities to bind to the target, the best-binding functionality may be omitted and the procedure repeated by mixing together n-2 molecules, omitting each time the said best-binding functionality plus a further molecule. This further molecule will be a different one each time the procedure is repeated. Comparing the binding effects of the libraries created, the second best functionality can be identified. Simultaneous removal thus allows for the identification of components which may contribute to activity but less that the optimal one(s).

If two sets of different molecules, nₓ and my (wherein nₓ=n₁, n₂, n₃, . . . nₓ and m_{y} is m₁, m₂, . . . m_{y}; x and y are integers ≧1) are mixed together for generation of a DCL of entities, a matrix of nₓ×m_{y} entities is created, each entity consisting of a molecule n of the set nₓ and a molecule m of the set m_{y}. An example might be the mixture of 12 aldehydes (aldehydes 1, 2, . . . 12) with 6 amines (amines A, B, C, D, E, F), so that 6×12=72 imines can be generated. Then the generation of the DCL is repeated as described above: In each repeating step another of the nₓ molecules or my molecules is omitted with the restriction that at least one molecule of each set must be present in the mixture. (No reaction could take place if no amine or no aldehyde were present in the mixture.) After screening e.g. for the best inhibitor as described above one or both of the two components forming the entity—one aldehyde and one amine—call be omitted and the procedure repeated by mixing together nₓ₋₁ and m_{y-2} or nₓ₋₂ and m_{y-1} or nₓ₋₂ and m_{y-2} molecules. There also results a ranking of the inhibitors.

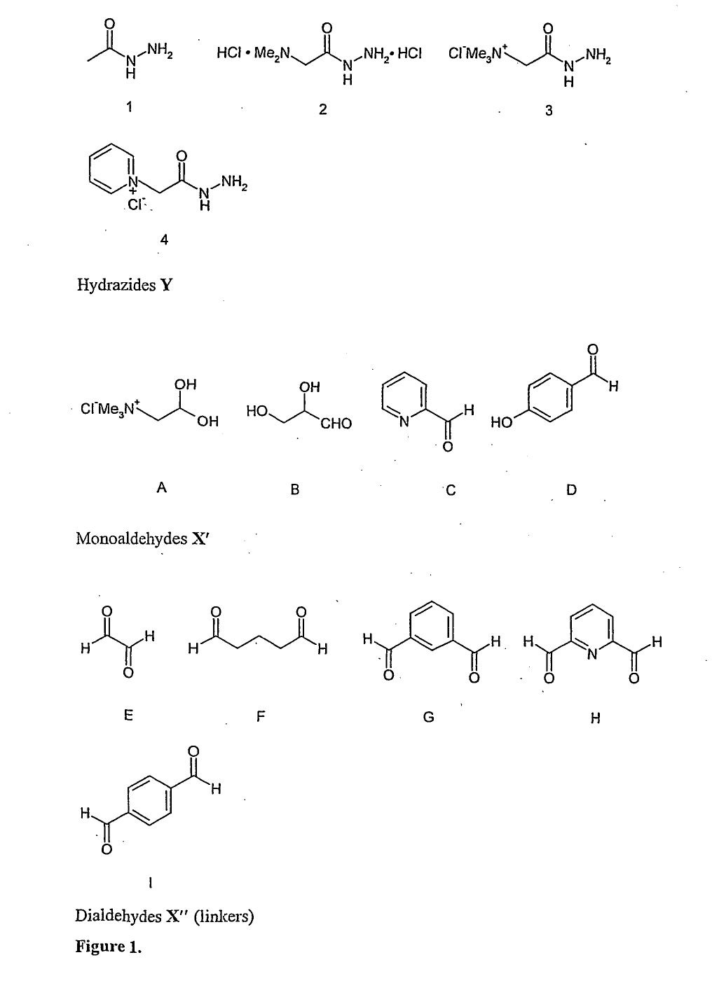

One may also omit more than one molecule in one step. Referring to Example 1 and FIG. 1, one can e. g. omit the aldehydes 1, 2 and 3 in the first repeating step, the aldehydes 4, 5 and 6 in the second step and so on, so every time 9 aldehydes and 6 amines would be mixed together. That means that the DCL generation is repeated n/z-times=12/3-times=four times.

It is also possible to omit the aldehydes 1, 2 and 3 in the first repeating step, the aldehydes 1, 2 and 4 in the second repeating step and so on, so that every time a new combination of three aldehydes is omitted. The same can be done with the anines, which would even accelerate the process. A huge amount of varieties, combinations and permutations is therefore possible and may be chosen by a person skilled in the art.

The method according to the present invention is preferably used when a dynamic combinatorial library is generated. These libraries are also an object of the present invention. However, the method can also be used when a static combinatorial library is generated.

In the case of a dynamic combinatorial library, the molecules generated generally must contain at least two functionalities. These functionalities are present in the starting molecules, and the library is formed by reversibly connecting these functionalities. The functionalities may be connected by a simple chemical bond, or by a group formed by functional groups present in the molecules which can reversibly be cleaved and formed. In the latter case, the starting molecules must contain at least two functionalities, one functionality for binding to the target, the other functionality being the functional group for the generation of the (spacer) group linking the functionalities which bind to the target.

When the n starting compounds to create the library are selected, there can be selected more than 10, preferably more than 100, most preferably more than 1000 molecules with differing functionalities. For example, there can be selected a set of 10 components with at least two functionalities with one, functionality being fixed and the other being varied. The fixed one serves to form a bond to another component, the other for binding to the target. One example is a set of molecules containing a carbohydrate head group, which differs from molecule to molecule, and a mercapto group. These mercapto groups can react with each other by forming a disulphide bond, so that entities result with two carbohydrate head groups which can bind to a target.

A further example are molecules which contain two or more target-binding functionalities which are separated by a spacer group allowing an exchange of the functionalities. This type of molecules can be used as starting materials for the generation of a dynamic combinatorial library. Examples include molecules with two carbohydrate head groups and an appropriate spacer-group between these like, for example, a spacer group having a disulphide bond or, referring to Example 2, a spacer group having two or more hydrazide functional groups which can react with functional groups present on the molecules carrying the functionalities, e.g. aldehyde groups.

Generally all molecules comprising atoms or functional groups between the functionalities which allow for a reversible formation and cleavage of bonds can be used as starting materials.

In another embodiment of the present invention two sets of differing molecules n and m (with n and m being integers ≧1) with at least two functionalities are selected. The selection is such that these molecules have comparable reactivities to avoid bias in the competition and a structural variability involving differing combinations of polar, nonpolar, and charged subunits. Each member of the set of m molecules can combine/react/connect with each member of the set of n molecules. This combination/connection can be a real covalent binding or any kind of interaction. So one of the functionalities in the molecules is used for reaction/connection with the molecules of the other set (i.e., this “functionality” in this case has the function of what has been defined beforehand as “functional group”, namely a group which serves to connect the molecule carrying the functionality to another such molecule) and the other functionality is used for binding to the target.

Examples of such two sets are aldehydes which have a second functional group like a sulfo group, a sulfonato group, a carboxy group or a sulfamoyl group and amines which have also a second functional group like a carboxy or amino group. The aldehydes can condensate with the anines to imines which present “entities” able to bind to a target.

Another example are two sets are aldehydes with a second functional group and hydrazides with a second functional group. They can condensate to “acyl hydrazones”. These second functional groups are e.g. selected from —NMe₂ and —⁺NMe₃ groups, hydroxy groups, pyridyl and phenyl groups and derivatives thereof.

As one set of molecules, bifunctional or oligofunctional molecules can be used, which act as linker to connect two or more molecules of the other set of molecules. Preferably, these molecules are symmetric, and they can have functional groups which are identical or different of each other, with identical functional groups being preferred. Examples for bifunctional molecules include e.g. dialdehydes, diamines, dicarbonic acids, disulfonamides, disulfonic acids, diols, dithiols,—preferably having two identical functional groups.

If the binding sites of the target are already known, molecules which contain the necessary functionalities to bind to the target are preferably selected. If one ligand for the target is already known, the molecules are preferable selected so that this ligand is mimicked.

In one preferred embodiment of the present invention the DCC/VCL concept is implemented to identify inhibitors of acetylcholinesterase (AChE) of a pre-equilibrated dynamic combinatorial library by dynamic deconvolution, see Example 1 below.

This is illustrated in FIGS. 1 to 3 which have the following meaning:

FIG. 1: Components X′, X″ and Y chosen as components for preparing the constituents of the pre-equilibrated dynamic combinatorial library of AChE inhibitors.

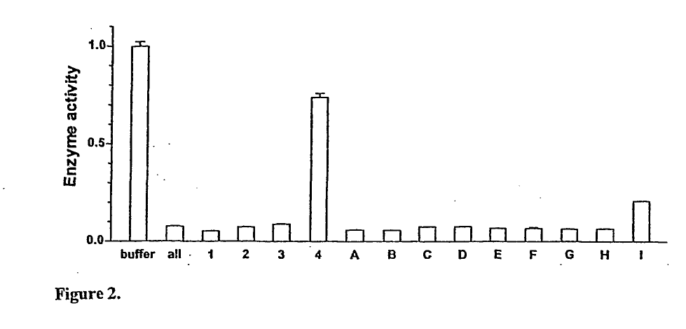

FIG. 2: Dynamic deconvolution of the full dynamic combinatorial library formed in the pool of all X and Y components; each bar corresponds to the removal of a given component 1-4 or A-I from the full library. The final concentration of each component in Ellman's test is 2×10⁻⁶ M for monofunctional (1-4 and A-D) and 1×10⁻⁶ M for bifunctional (E-I) components.

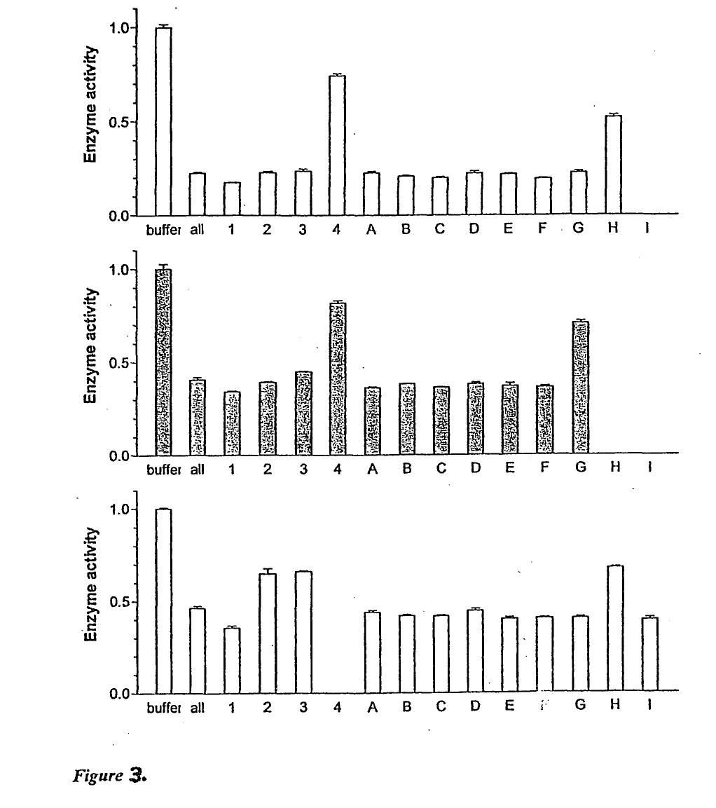

FIG. 3: Sequential dynamic deconvolulion of the dynamic combinatorial library. Dynamic deconvolution of the initial full library containing 1-4 and A-I (FIG. 2): (top) a library containing 1-4 and A-H; (center) a library containing 1-4 and A-G; (bottom) a library containing 1-3 and A-I.

AChE is an enzyme whose function in the central and peripheral nervous system it is to terminate transmission at cholinergic synapses by hydrolysing the cationic neurotransmitter acetylcholine (ACh). Its inhibitors might therefore be of interest for the treatment of the Alzheimer's disease.

A dynamic combinatorial library (DCL) can be generated through reversible “acyl hydrazone” connections (in acidic aqueous medium) between hydrazide and monoaldehyde or dialdehyde component precursors (FIG. 1). The formation and component interchange process is rapid in acidic aqueous condition and rather slow in neutral and basic conditions so that the reaction ban be easily controlled by adjusting the pH.

Equilibration within the pool of components can, for example, be performed in an acetate buffer where the formation and interchange is rapid, at a higher pH the inter-conversion of the library constituents is blocked.

In order to identify the active constituent(s), one of the components in the pool library was sequentially omitted.

The results are presented in FIG. 2: The complete pool library is generated by adding all components (1-4, A-I, see structures in FIG. 1) under pre-equilibrating conditions (acetate buffer pH 4.0). The sub-libraries are formed by mixing all components except a specific X or Y component under the same conditions.

The complete pool library is composed of all possible condensation products in proportion to their relative thermodynamic stability. After scrambling has been stopped, the inhibition of the AChE activity by the libraries were measured. “Buffer” represents the AChE activity in the absence of any inhibitor. The data in FIG. 2 show that the largest effects are observed when either 4 or I have been removed from the pool. Consequently, the most active constituent must come from the assembly of fragments 4 and I, which is in agreement with the results obtained from the separate investigation of individual compounds.

FIG. 3 illustrates the results of sequential removal of a component X or Y from a pool which does not contain I (top), or 4 (bottom) or neither H nor I (center). It is revealed that G, H, 2 and 3 represent also active fragments with a relative activity sequence G≦H≦I and 2˜3<4. One may also infer that the connection/combination (143) must have substantial activity. In addition, although I4₂ is more active than H4₂, the data reveal that in absence of 4, H yields more active connection/combinations with 2 or 3 than does I.

In a further preferred embodiment, the method according to the present invention is applied to a DCL formed from the reaction between anines and aldehydes carrying functionalities which may bind to a target. There are thus reversibly generated imines containing target-binding functionalities. One example is disclosed in the above-cited Proc.Natl.Acad. Sci. USA 1997, 94, 2106-2110, relating to amines and aldehydes containing functionalities which may bind to carbonic anhydrase. The method of creating a DCL and the starting compounds disclosed in the above reference are an integral part of the present invention and herein incorporated by reference.

In still a further preferred embodiment, the method according to the present invention is applied to a DCL formed from the scrambling of carbohydrates, which may bind to a target, around a spacer group. There are thus reversibly generated molecules containing two or more target-binding functionalities. One example is disclosed in the above-cited application PCT/EP01/02310 of the applicant, relating to pentoses and hexoses containing functionalities which may bind to lectins, in particular Concanavalin A, with the spacer group containing a disulfide bond. The method of creating a DCL and the starting compounds disclosed in the above-cited application are an integral part of the present invention and herein incorporated by reference. In Example 2 of the present invention the DCC/VCL concept is implemented to identify ligands to the lectin Concanavalin A (Con A) of a pre-equilibrated dynamic combinatorial library by dynamic deconvolution, wherein the spacer group contains hydrazide functionalities.

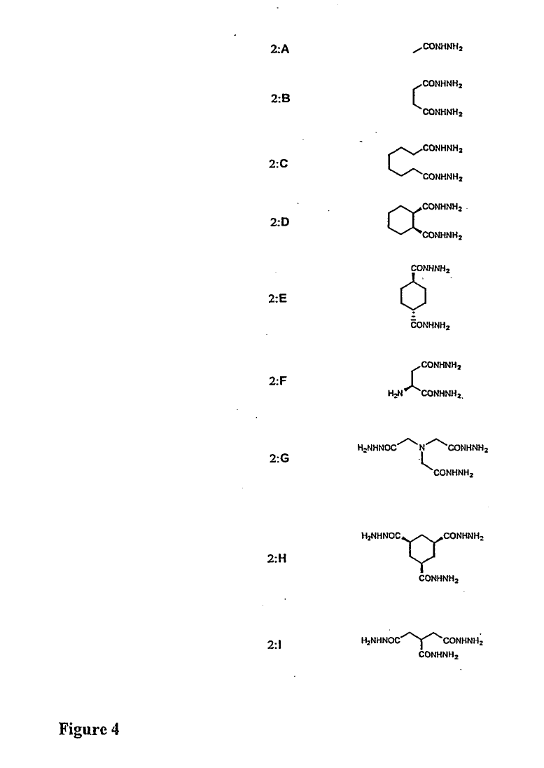

This is illustrated in FIGS. 4 to 6 which have the following meaning:

FIG. 4: Components 2:1-6, 2:A-I chosen as components for preparing the constituents of the pre-equilibrated dynamic combinatorial library of Con A ligands.

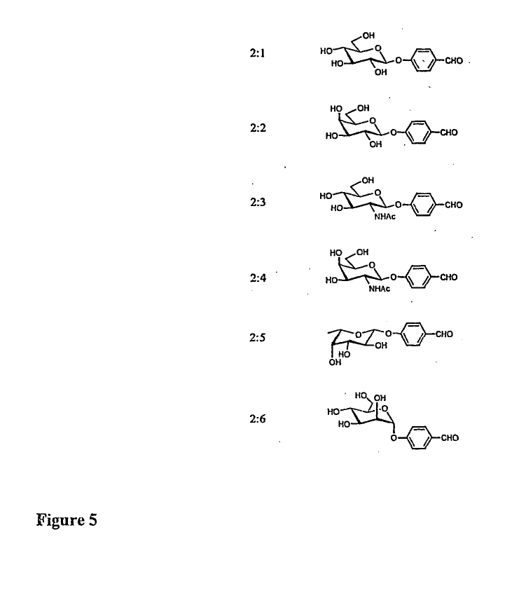

FIG. 5: Dynamic deconvolution of the full dynamic combinatorial library formed in the pool of all components; each bar corresponds to the removal of a given component 2:1-6 or 2:A-I from the full library.

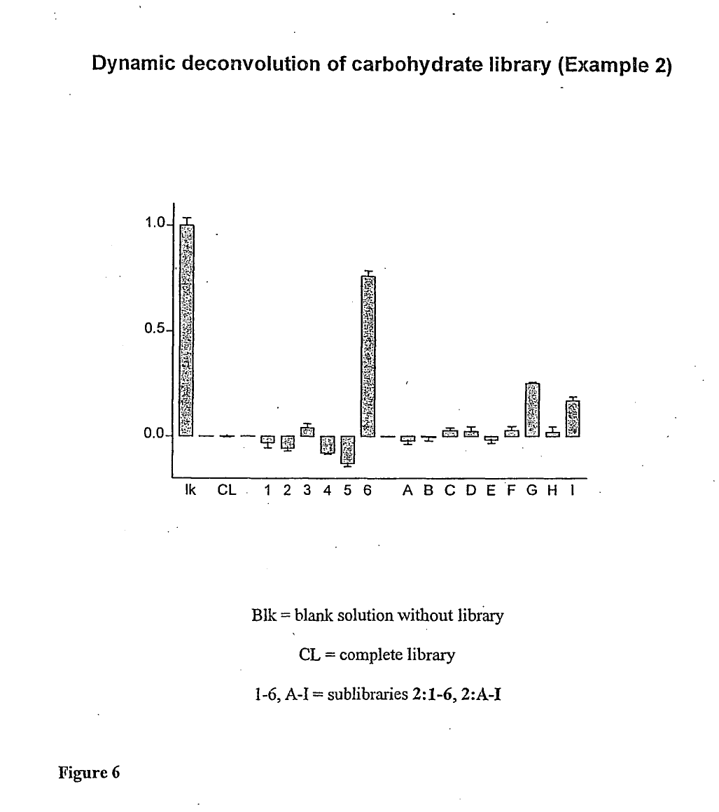

FIG. 6: Structure of the resulting Con A ligand emanating from the dynamic deconvolution procedure.

The results are presented in FIG. 5: The complete pool library is generated by adding all components (2:1-6, 2:A-I, see structures in FIG. 4) under pre-equilibrating conditions (ammonium formate buffer pH 4.0). The sub-libraries are formed by mixing all components except a specific component under the same conditions.

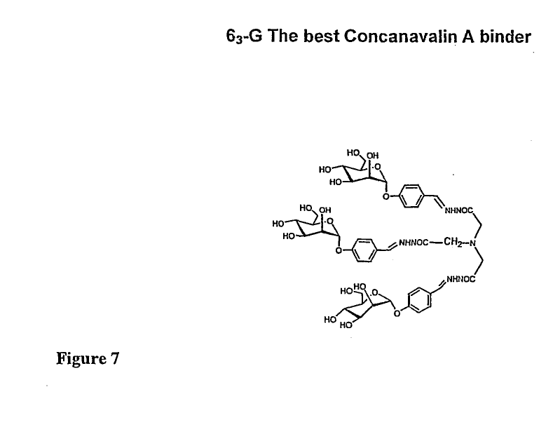

The complete library (CL) is composed of all possible condensation products in proportion to their relative thermodynamic stability. After scrambling has been stopped, the binding to Con A by the libraries was measured. “Blk” represents the maximal signal in the absence of any ligand (blank). The data show that the largest effects are observed when either 6 or G have been removed from the pool. Consequently, the most active constituent comes from the assembly of fragments 6 and G (FIG. 7).

### EXAMPLES

### Example 1

General Procedures

Acetylcholinesterase (AChE; from Torpedo Marmorata) was purified as described. Acetylthiocholine iodide was purchased from Sigma. DTNP [5,5′-dithiobis(2-nitrobenzoic acid), Ellman's reagent] was obtained from Acros. Formyltetramethylammonium chloride (betainealdehyde chloride) was synthesized from dimethylaminoacetaldehyde diethyl acetal. The other reagents were purchased from commercial sources and used without further purification. Enzyme assays were carried out using a Varian Cary 3 UV-Visible spectrophotometer.

Synthesis of Formyltetramethylammonium Chloride

Methyl iodide (0.93 mL, 14.9 mmol) was added dropwise to a solution of dimethylaminoacetaldehyde diethyl acetal (2.27 mL, 12.4 mmol) in 30 mL MeOH. The mixture was refluxed for 5 h and then the solvent was removed. The residue was dissolved in 20 mL of water and then silver iodide (2 g) was added to change the ammonium iodide salt to the ammonium chloride salt. The suspension was filtered and the solvent was removed from filtrate under vacuum. The product was recrystallized from ethanol and diethyl ether yielding a white solid. The white solid from the former reaction was dissolved in 10% aq. HCl (20 mL). The mixture was heated at 60° C. overnight. The solvent was removed and the product was recrystallized from formic acid and formaldehyde.

Formation of X′Y and X″Y₂ Connections

20 μL of a solution of X (50 mM of monoaldehyde X′ or 25 mM of dialdehyde X″) and 20 μL of a solution of Y (50 mM of monohydrazide) was added to sodium acetate buffer at pH 4,0 (460 μL of 100 mM solution). The mixture was equilibrated at ambient temperature for one week to ensure full reaction. However, less time, from about 15 min to 2 days, depending on the case, may be needed. An aliquot of the solution was taken to test the inhibitory activity on AChE by Ellman's method. Note: The concentration of X′Y is twice that of X″Y₂.

The solutions of A, B (50 mM) and E, F (25 mM) were prepared in 100 mM sodium acetate buffer, pH 4.0. The solutions of C, D (50 mM) and G, I (25 mM) were prepared in 12.5 % CH₃CH/100 mM sodium acetate buffer, pH 4.0. The solution of H (25 mM was prepared in 16.7% CH₃CN/100 mM sodium acetate buffer, pH 4.0.

The solutions of 1-4 (50 mM) were prepared in 100 mM sodium acetate buffer, pH 4.0.

Generation of the Pre-Equilibrated Dynamic Combinatorial Libraries for the Deconvolution Experiments (FIG. 2)

Full library (all): 20 μL of a solution of each component (1-4 and A-I, mM for monofunctional and 2.5 mM for bifunctional compounds) were added to 240 μL of 100 mM sodium acetate buffer at pH 4.0. The final volume was 500 μL. The equilibrating mixture was stirred at ambient temperature.

Partial library (X or Y): 20 μL of a solution of each component (1-4 and A-I, 5 mM for monofunctional and 2.5 mM for bifunctional compounds) except for the X or Y compound to be omitted, were added to 260 μL of 100 mM sodium acetate buffer at pH 4.0 to make the final volume 500 μL. The equilibrating mixture was stirred at ambient temperature.

In both cases equilibration was conducted for one week. However, reaction times may be much shorter (from about 15 min to 2 days) depending on the components.

The solutions of A, B, 1-4 (5 mM) and E, F (2.5 mM) were prepared in 100 mM sodium acetate buffer, pH 4.0. The solutions of C, D (5 mM) and G, I (2.5 mM) were prepared in 1.25% CH₃CN/100 m M sodium acetate buffer, pH 4.0. The solution of H (2.5 mM) was prepared in 1.67% CH₃CN/100 mM sodium acetate buffer, pH 4.0.

Enzyme Assay

The inhibitory activity of the equilibrated mixtures was determined by using. Ellman's method. 10 μL of a 50 mM solution of acetylthiocholine iodide in water and 10 μL of a solution of an equilibrated mixture as prepared above or of blank pH 4.0 solution were added to 980 μL of a solution of 1 mg/mL DTNP in 50 mM sodium, potassium phosphate buffer at pH 7.2. The activity of AChE was monitored by following the change in absorbance (V=ΛΔmin) at 412 nm over 30 seconds at 25° C. Inhibition is given by the equation:

$$\% inhibition = \frac{{V}_{0} - {V}_{i}}{{V}_{0}} \times 100 ,$$

where V₀ is the enzyme activity of blank solution (100 mM sodium acetate buffer, pH 4.0 without inhibitor) and Vᵢ is the enzyme activity of the solution in presence of inhibitor.

### Example 2

General Procedures

Concanavalin A and all reagents were obtained from common commercial sources (Lancaster, Fluka, Aldrich, Bachem, Sigma). Dichloromethane was distilled from calcium hydride prior to use. The binding effect of the equilibrated mixtures was determined using competitive inhibition of enzyme-labelled concanavalin A, and were carried out using a Victor microtiter plate reader.

Synthesis of Hydrazides 2A-2I

The hydrazides (2A-2I) were prepared from the corresponding carboxylic methyl esters, the latter synthesised by treating the carboxylic acids with methanol under acidic conditions. In a typical example: Cyclohexane cis-, cis-, 1,3,5-tricarboxylic acid (2.0 g, 9.3 mmol) was dissolved in methanol (100 mL), sulfuric acid (2 mL) was added, and the reaction allowed to proceed at room temperature overnight. The solution was concentrated, diluted with dichloromethane, and washed with aqueous bicarbonate, water, and brine. After drying, the crude methyl ester was dissolved in methanol (150 mL), hydrazine hydrate (3 mL, 45 mmol) was added and the reaction left at room temperature overnight. The resulting precipitate was filtered off, washed with methanol and dried in vacuo (80%).

Synthesis of Carbohydrate Aldehydes (2:1-6)

Carbohydrate aldehydes 2:1-5 were synthesised from the corresponding peracetylated species following published procedures.

Carbohydrate Aldehyde 2:6

1,2,3,4,6-O-pentaacetyl-D-mannoside (2.5 g, 6.4 mmol) was dissolved in HBr/HOAc (125 mL, 33% v/v in HOAc) and the reaction allowed to proceed at ambient temperature for 4 h. The resulting solution was poured on ice, extracted with dichloromethane, washed with aqueous NaHCO₃, water, brine, dried over Na₂SO₄, concentrated and dried in vacuo. The product was redissolved in dry dichloromethane (100 mL), to which was added calcium sulphate (3.0 g), 4-hydroxybenzaldehyde (1.6 g, 12.8 mmol), and silver triflate (3.3 g, 12.8 mmol). After completed reaction at ambient temperature (3 h), the product mixture was filtered through celite, neutralised with 1 M NaHSO₄ (30 mL), washed with water and brine, dried and concentrated. The organic phase was washed with 1 M NaOH, water, brine, and dried over Na₂SO₄. Flash chromatography (SiO₂; hexane/ethyl acetate 1:1 v/v), concentration and drying yielded a white foam (25%), which upon deprotection under standard Zemplén conditions (NaOMe in MeOH) yielded an off-white foam (quant.).

Generation of Acyl Hydrazone Libraries

The complete library (CL) was designed to contain near equal amounts of each final species at a concentration of approximately 12 μM. The final solution contained 2.5 mM of each carbohydrate aldehyde (2:1-6), and varying amounts of hydrazides: 2:A, 70 μM; 2:B-E, 0.24 mM; 2:F, 0.42 mM; 2:G, 0.65 mM; 2:H, 0.88 mM; 2:I, 2.5 mM. The mixture was made in ammonium formate buffer at pH 4.0, and was equilibrated at ambient temperature for one week to ensure full reaction.

The different sublibraries (SL1-SLI) were prepared under identical conditions, where one component (2:1-6, 2:A-I) was removed from the mixture each time. Aliquots of the solution were subsequently taken out to test the inhibitory activity on Concanavalin A.

1. A method for the generation of a dynamic combinatorial library of ligands for a given target, capable of binding at least two functionalities, which method comprises the following steps:
(i) selecting a set of n molecules carrying functionalities which may bind to the target, which molecules are capable of reversibly binding to each other under formation of an entity, n being an integer ≧1;
(ii) mixing together said set of said n molecules;
(iii) subjecting the mixture to conditions allowing a reversible bond formation and cleavage under formation of entities carrying different combinations of functionalities, until equilibrium is reached;
(iv) optionally transforming the said entities into ligands;
(v) repeating steps (i) to (iv) n-times, each time a different molecule of said set of n molecules being omitted;
(vi) assessing the ability of each of the mixtures obtained to bind to a given target.

2. A method for the generation of a dynamic combinatorial library of ligands for a given target, capable of binding at least two functionalities, which method comprises the following steps:
(i) selecting a set of n molecules carrying functionalities which may bind to the target, n being an integer ≧1, and linking said functionalities by a spacer group, hence creating a set of entities carrying different combinations of said functionalities;
(ii) mixing together said set of entities;
(iii) subjecting the mixture to conditions allowing a reversible bond formation and cleavage of or at the spacer group, hence a scrambling of the functionalities under the formation of new entities;
(iv) optionally transforming said entities generated to ligands;
(v) repeating steps (i) to (iv) several times, each time a different functionality of said n functionalities being omitted;
(vi) assessing the ability of each of the mixtures obtained after carrying out the above steps to bind to a given target.

3. The method according to claim 1 or 2, wherein the mixture obtained after step (iii) is subjected to conditions which stop the process of bond formation and cleavage, before the step (v) is carried out with the said molecules or functionalities.

4. The method according to any of the claims 1 to 3, wherein in step (v) more than one molecule or functionality of said set of n molecules or n functionalities is omitted each time the steps (i) to (iv) are repeated.

5. The method according to any of the claims 1 to 3, comprising a further step (vii) wherein the steps (i) to (iv) are repeated, each time the most active molecule or functionality plus at least one further molecule or functionality being omitted.

6. The method according to any of the claims 1 to 5, wherein the mixtures obtained in the respective steps are analyzed and the results compared.

7. The method according to any of the claims 1 to 6, wherein the functionality is selected from the group consisting of amino and imino groups and derivatives thereof, hydroxy and mercapto groups and derivatives thereof, oxo and thioxo groups, formyl and thioformyl groups, aryl groups, substituted aryl groups, phenyl groups, substituted phenyl groups, pyridyl groups and derivatives thereof, carboxy groups and carboxylato groups and derivatives therof, alkyloxycarbonyl groups, (di)thiocarboxy groups and derivatives thereof, (di)thiocarboxylato groups, carbamoyl groups and derivatives thereof, sulfo, sulfino and sulfeno groups and derivatives thereof, alkyloxysulfonyl, alkyloxysufinyl and alkyloxysulfenyl groups, sulfamoyl, sulfinamoyl and sulfenamoyl groups and derivatives thereof, cyano and (iso)(thio)cyanato groups, hydroperoxy groups, nitroso groups, hydroxyamino groups, hydrazino groups, —NR₁R₂, —⁺NHR₁R₂ and −⁺NR₁R₂R₃ groups, wherein R₁, R₂, and R₃ are identical or different and represent alkyl, cycloalkyl, alkylcycloalkyl, aryl, alkylaryl with 1 to 40 C atoms, −+OR₁R₂ groups wherein R₁ and R₂ are identical or different and represent alkyl, cycloalkyl, alkylcycloalkyl, aryl, alkylaryl with 1 to 40 C atoms, hydrazide groups, heterocycles carrying one or more heteroatoms in the ring selected from the group consisting of N, O and S, amino acids and oligo- and polypeptides, sugars, sugar derivatives and oligomers and polymers therof, and nucleic acids and derivatives thereof.

8. The method according to claim 7 wherein the functionality is a sugar, the sugar being preferably selected from the group consisting of hexoses and pentoses.

9. The method according to any of the claims 1 to 8, where the molecules carrying the functionalities carry themselves functional groups selected from amino groups, aldehyde groups, keto groups, thiol groups, olefinic groups, alcohol group, carbonyl groups, hydrazine groups, hydroxylamine groups and borate groups.

10. The method according to any of the claims 1 to 9, wherein the at least two functionalities are linked by a group selected from the group consisting of amines, acetals, oximes, esters, alkenes, imines, acylhydrazones and disulphides.

11. The method according to claim 10, wherein the reversible bond formation and cleavage is stopped and initiated by adjusting the pH of the solution.

12. The method according to any of claims 1 to 11, wherein the target is a protein, an enzyme, a biological receptor or an antibody.

13. The method according to claim 12, wherein the said target is a carbohydrate binding protein, preferably a lectin, in particular Concanavalin A.

14. The method according to claim 12, wherein the said target is carbonic anhydrase or acetylcholinesterase.

15. The method according to claim 12, wherein the said ligand is selected from the group of a substrate, in particular an activator and an inhibitor of said target, said target being an enzyme or an analogue thereof.

16. The method according to claim 12, wherein said ligand is selected from the group of agonists and antagonists of a receptor.

17. The method according to claim 12, wherein said ligand is an antigen, said target being an antibody.

18. A dynamic combinatorial library of ligands which is obtainable by the method is according to any of the claims 1 to 17

19. The library according to claim 18, wherein the said ligand is an inhibitor.

20. A molecule obtainable by the method according to any of the claims 1 to 16.
